(19) United States
(12) Patent Application Publication
(10) Pub. No.: US 2006/0224232 A1
(43) Pub. Date: Oct. 5, 2006

(54) Hybrid modular endovascular graft

(75) Inventor: Michael V. Chobotov, Santa Rosa, CA (US)

Correspondence Address: HOFFMANN & BARON, LLP, 6900 JERICHO TURNPIKE, SYOSSET, NY, 11791, US

(73) Assignee: TriVascular, Inc., Santa Rosa, CA (US)

(21) Appl. No.: 11/097,718

(22) Filed: Apr. 1, 2005

(51) Int. Cl. A61F 2/06

(52) U.S. Cl. 623/1.16

(57) ABSTRACT

A hybrid modular endovascular graft wherein a main graft is sized to span at least a portion of a target vessel lesion in a large percentage of patients. Graft extensions may be secured to the main graft to extend the main graft and provide a sealing function for some applications.

### BACKGROUND OF THE INVENTION

An aneurysm is a medical condition indicated generally by an expansion and weakening of the wall of an artery of a patient. Aneurysms can develop at various sites within a patient's body. Thoracic aortic aneurysms (TAAs) or abdominal aortic aneurysms (AAAs) are manifested by an expansion and weakening of the aorta which is a serious and life threatening condition for which intervention is generally indicated. Existing methods of treating aneurysms include invasive surgical procedures with graft replacement of the affected vessel or body lumen or reinforcement of the vessel with a graft.

Surgical procedures to treat aortic aneurysms can have relatively high morbidity and mortality rates due to the risk factors inherent to surgical repair of this disease, as well as long hospital stays and painful recoveries. This is especially true for surgical repair of TAAs, which is generally regarded as involving higher risk and more difficulty when compared to surgical repair of AAAs. An example of a surgical procedure involving repair of a AAA is described in a book titled Surgical Treatment of Aortic Aneurysms by Denton A. Cooley, M.D., published in 1986 by W. B. Saunders Company.

Due to the inherent risks and complexities of surgical repair of aortic aneurysms, endovascular repair has become a widely used alternative therapy, most notably in treating AAAs. Early work in this field is exemplified by Lawrence, Jr. et al. in “Percutaneous Endovascular Graft: Experimental Evaluation”, Radiology (May 1987) and by Mirich et al. in “Percutaneously Placed Endovascular Grafts for Aortic Aneurysms: Feasibility Study,” Radiology (March 1989). Commercially available endoprostheses for the endovascular treatment of AAAs include the AneuRx® stent graft system manufactured by Medtronic, Inc. of Minneapolis, Minn., the Zenith® stent graft system sold by Cook, Inc. of Bloomington, Ind., the PowerLink® stent graft system manufactured by Endologix, Inc. of Irvine, Calif., and the Excluder® stent graft system manufactured by W.L. Gore & Associates, Inc. of Newark, Del. A commercially available stent graft for the treatment of TAAs is the TAG™ system manufactured by W.L. Gore & Associates, Inc.

When deploying such endovascular devices by catheter or other suitable instrument, it is advantageous to have a flexible and low profile stent graft and delivery system for passage through the various guiding catheters as well as the patient's sometimes tortuous anatomy. Many of the existing endovascular devices and methods for treatment of aneurysms, while representing significant advancement over previous devices and methods, use systems having relatively large transverse profiles, often up to 24 French. Also, such existing systems have greater than desired longitudinal stiffness, which can complicate the delivery process. In addition, the sizing of stent grafts may be important to achieve a favorable clinical result. In order to properly size a stent graft, the treating facility typically must maintain a large and expensive inventory of stent grafts in order to accommodate the varied sizes of patient vessels due to varied patient sizes and vessel morphologies. Alternatively, intervention may be delayed while awaiting custom size stent grafts to be manufactured and sent to the treating facility. As such, non-invasive endovascular treatment of aneurysms is not available for many patients that would benefit from such a procedure and can be more difficult to carry out for those patients for whom the procedure is indicated. What has been needed are stent graft systems and methods that are adaptable to a wide range of patient anatomies and that can be safely and reliably deployed using a flexible low profile system.

### BRIEF SUMMARY OF THE INVENTION

An embodiment of a hybrid modular endovascular graft system includes a main graft having a main fluid flow lumen therein, a distal leg having a fluid flow lumen therein, a proximal anchor member disposed at a proximal end of the main graft and a distal anchor member disposed on a distal portion of the distal leg. The distal anchor member is axially separated from the proximal anchor member by a distance of about 12.0 cm to about 14.0 cm. The graft system also includes a graft extension having a fluid flow lumen disposed therein. The fluid flow lumen of the graft extension is overlapped and in fluid communication with the fluid flow lumen of the distal leg. In some embodiments, the main graft further comprises a network of inflatable channels distributed over a main graft body section and distal leg to provide structural rigidity and support to the main graft when the network of inflatable channels are in an inflated state. In still other embodiments of the hybrid modular graft system, the main graft is configured as a bifurcated graft and further includes a second distal leg having a fluid flow lumen therein which is in fluid communication with the main fluid flow lumen. A second distal anchoring member is disposed on a distal portion of the second distal leg. Such hybrid modular graft system embodiments may also include a second graft extension having a fluid flow lumen disposed therein which may be deployed with the fluid flow lumen of the second graft extension overlapped and in fluid communication with the fluid flow lumen of the second distal leg.

In an embodiment of a method of treating the vasculature of a patient, a hybrid modular graft system is provided. The hybrid modular graft system includes a main graft having a main fluid flow lumen therein, a distal leg having a fluid flow lumen disposed therein, a proximal anchor member disposed at a proximal end of the main graft and a distal anchor member disposed at a distal end of the distal leg. The distal anchor member is axially separated from the proximal anchor member by a distance of about 12.0 cm to about 14.0 cm. The graft system also includes a graft extension having a fluid flow lumen disposed therein which is sealable to the fluid flow lumen of the distal leg. Once the graft system has been provided, the main graft is positioned within the patient's vasculature and the proximal anchor member anchored in the patient's aorta and the distal anchor member anchored in an iliac artery of the patient. The graft extension is positioned relative to the distal leg of the main graft such that the fluid flow lumen of the graft extension is overlapped and in fluid communication with the fluid flow lumen of the distal leg. The graft extension may then be deployed.

### BRIEF DESCRIPTION OF THE DRAWINGS

FIG. 1 is an elevational view of a hybrid modular graft system including an inflatable main graft and a graft extension.

FIG. 2 is a transverse cross section of the hybrid modular graft system of FIG. 1 taken along lines 2-2 of FIG. 1.

FIG. 3 is a transverse cross section of the hybrid modular graft system of FIG. 1 taken along lines 3-3 of FIG. 1.

FIG. 4 is a transverse cross section of the graft extension of the hybrid modular graft system of FIG. 1 taken along lines 4-4 of FIG. 1.

FIG. 5 shows the main graft of FIG. 1 deployed within an abdominal aortic aneurysm of a patient with the proximal anchor member, ipsilateral distal anchor member and contralateral distal anchor member of the main graft secured to the inside of the patient's vasculature.

FIG. 6 illustrates the main graft of FIG. 5 with the graft extension deployed such that the fluid flow lumen of the graft extension overlaps the fluid flow lumen of the first distal leg of the main graft.

FIG. 6A is an enlarged view in partial section of the encircled portion 6A-6A in FIG. 6.

FIG. 7 is an elevational view of a graft system including a non-inflatable main graft and graft extension.

FIG. 8 is a transverse cross section of the hybrid modular graft system of FIG. 7 taken along lines 8-8 of FIG. 7.

FIG. 9 is a transverse cross section of the hybrid modular graft of FIG. 7 taken along lines 9-9 of FIG. 7.

FIG. 10 is a transverse cross section of the graft extension of the hybrid modular graft system of FIG. 7 taken along lines 10-10 of FIG. 7.

FIG. 11 shows the main graft of FIG. 7 deployed within an abdominal aortic aneurysm of a patient with the proximal anchor member, ipsilateral distal anchor member and contralateral distal anchor member of the main graft secured to the inside of the patient's vasculature.

FIG. 12 illustrates the main graft of FIG. 11 with the graft extension deployed such that the fluid flow lumen of the graft extension is overlapped with the fluid flow lumen of the first or ipsilateral distal leg of the main graft.

FIG. 12A is an enlarged view in partial section of the encircled portion 12A-12A in FIG. 12.

### DETAILED DESCRIPTION OF THE INVENTION

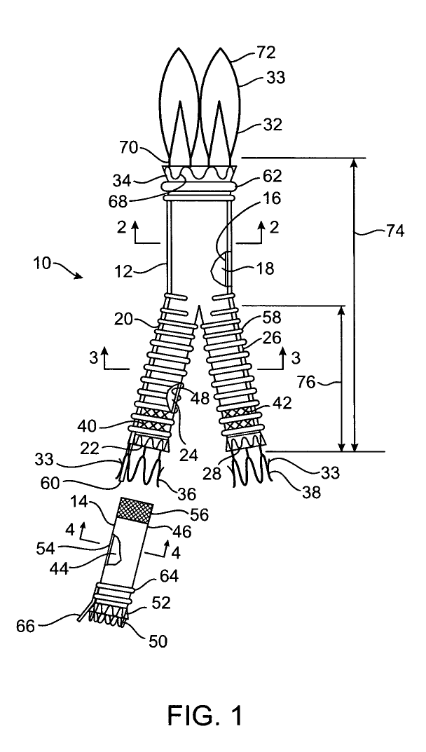

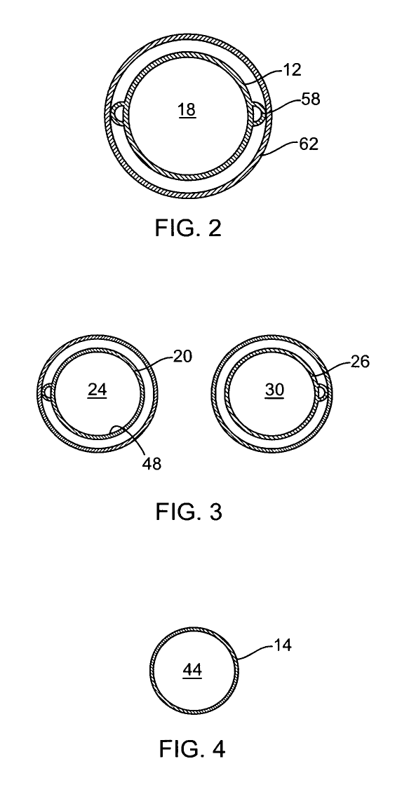

Embodiments of the invention are directed generally to methods and devices for treatment of fluid flow vessels with the body of a patient. Treatment of blood vessels is specifically indicated for some embodiments, and, more specifically, treatment of abdominal aortic aneurysms for others. FIGS. 1-4 show a bifurcated embodiment of a hybrid modular graft system 10 for treatment of an abdominal aortic aneurysm. The graft system includes a bifurcated main graft 12 and an ipsilateral graft extension 14. The main graft 12 has a wall portion 16 that bounds a main fluid flow lumen 18 disposed therein. An ipsilateral leg 20 has a ipsilateral port 22 and an ipsilateral fluid flow lumen 24 that is in fluid communication with the main fluid flow lumen 18 and the ipsilateral port 22. A contralateral leg 26 has a contralateral port 28 and a contralateral fluid flow lumen 30 that is in fluid communication with the main fluid flow lumen 18 and the contralateral port 28. The main graft 12, ipsilateral leg 20 and contralateral leg 26 form a bifurcated “Y” shaped configuration with the main fluid flow lumen 18 of the main graft 12 typically having a larger transverse dimension and area than the fluid flow lumens 24 and 30 of either the ipsilateral leg 20 or contralateral leg 26. A proximal anchor member 32 is disposed at a proximal end 34 of the main graft 12. An ipsilateral distal anchor member 36 is disposed on the distal end of the ipsilateral leg 20. A contralateral distal anchor member 38 is disposed on the distal end of the contralateral leg 26. An optional ipsilateral attachment element 40 is disposed on a distal portion of the ipsilateral leg 20 and an optional contralateral attachment element 42 is disposed on a distal portion of the contralateral leg 26.

The graft extension 14 has a fluid flow lumen 44 disposed therein which is sized and configured to be sealed in fluid communication with the fluid flow lumen 24 of the ipsilateral leg 20. Typically, an outside surface 46 of the graft extension 14 will be sealed to an inside surface 48 of the ipsilateral leg 20 of the main graft 12 when the graft extension 14 is deployed. An extension anchor member 50 is secured to a distal end of the graft extension 14 or an ipsilateral connector ring 52 that is at least partially disposed in a wall portion 54 of the distal portion of the graft extension 14. The extension anchor member 50 may be in the form of an expandable member or stent. The extension anchor member 50 may be used to anchor the distal end of the graft extension 14 to the patient's vasculature. An optional first attachment element 56 is disposed adjacent a proximal end of the graft extension 14 and is configured to be securable to the ipsilateral attachment element 40 with the fluid flow lumen 44 of the graft extension 14 sealed to the fluid flow lumen 24 of the ipsilateral leg 20. The first attachment element 56 and ipsilateral attachment element 40 may, for example, be configured as any of the attachment elements in copending and commonly owned U.S. patent application Ser. No. _{——————}, entitled “Modular Endovascular Graft”, filed Mar. 11, 2005, by Vinluan et al. (Attorney Docket No. 21630-006810US), which is hereby incorporated by reference herein in its entirety.

The transverse dimension or diameter of the main fluid flow lumen 18 may be from about 15.0 mm to about 32.0 mm. The transverse dimension or diameter of the ipsilateral and contralateral fluid flow lumens 24 and 30 of the ipsilateral leg 20 and contralateral leg 26 may be from about 5.0 mm to about 20.0 mm. The length of the contralateral leg 26 is indicated by arrow 76 in FIG. 1. For one embodiment, the length of the legs 20 and 26 and can be from about 4.0 cm to about 10.0 cm. The transverse dimension of an embodiment of the graft extension 14 may be from about 5.0 mm to about 20.0 mm. The length of an embodiment of the graft extension 14 may be from about 2.0 cm to about 10.0 cm; specifically, about 5.0 cm to about 8.0 cm. The main graft 12 and ipsilateral graft extension 14 may be made from any suitable materials, including polytetrafluoroethylene (PTFE) or expanded polytetrafluoroethylene (ePTFE). In particular, main graft 12 and graft extension 14 may comprise any number of layers of PTFE and/or ePTFE, including from about 2 to about 15 layers, having an uncompressed layered thickness of about 0.003 inch to about 0.015 inch. Unless otherwise specifically stated, the term “PTFE” as used herein includes both PTFE and ePTFE. Furthermore, the graft body sections of the present invention described herein may comprise all PTFE, all ePTFE, or a combination thereof. Such graft body sections may comprise any alternative biocompatible materials, such as DACRON, suitable for graft applications. Descriptions of various constructions of graft body sections may be found in commonly-owned pending U.S. patent application Ser. No. 10/029,557, entitled “Method and Apparatus for Manufacturing an Endovascular Graft Section”, U.S. patent application Ser. No. 10/029,584, entitled “Endovascular Graft Joint and Method of Manufacture”, U.S. patent application Ser. No. 10/029,570, entitled “Method and Apparatus for Shape Forming Endovascular Graft Material” (now U.S. Pat. No. 6,776,604), and U.S. patent application Ser. No. 10/029,559, entitled “Advanced Endovascular Graft,” all of which were filed on Dec. 20, 2001 to Chobotov et al. and U.S. patent application Ser. No. 10/168,053, entitled “Inflatable Intraluminal Graft,” filed Jun. 14, 2002 to Murch, the entirety of each of which is incorporated herein by reference.

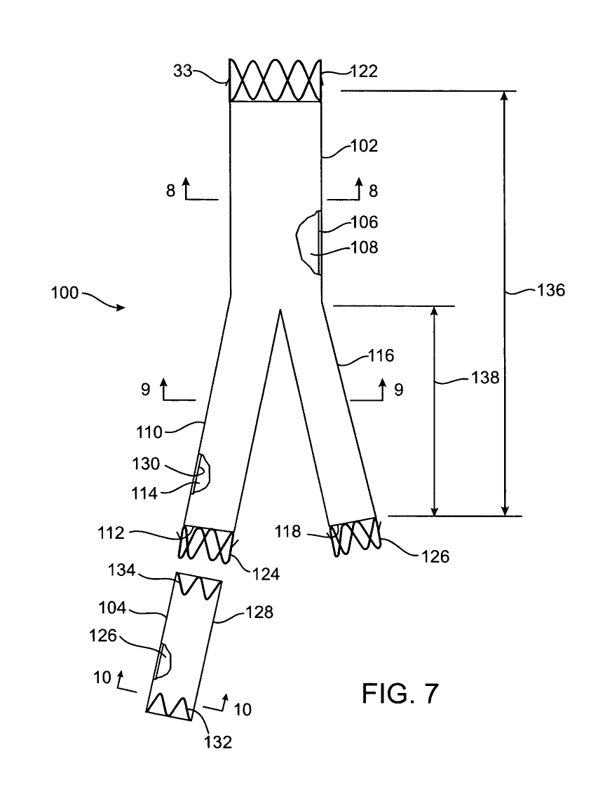

For embodiments of graft systems that do not include the attachment elements, the proximal end of the graft extension 14 may be expanded against the inside surface 48 of the fluid flow lumen 24 of the ipsilateral leg 20 to seal the fluid flow lumen 44 of the graft extension 14 to the fluid flow lumen 24 of the ipsilateral leg 20. Expandable members, such as expandable anchor members and the like, may be used to expand the graft extension 14 against the inside surface 48 of the fluid flow lumen 24 of the ipsilateral leg 20. Such embodiments are discussed in more detail below with regard to the non-inflatable hybrid graft system of FIG. 7. A second or contralateral graft extension (not shown) may have the same features as the ipsilateral graft extension 14 including a fluid flow lumen disposed therein and a distal anchor member disposed at a distal end of the second graft extension. An optional second attachment element disposed adjacent a proximal end of the second graft extension may be configured to be securable to the contralateral distal attachment element 42 on the contralateral leg 26 of the main graft 12.

A network of inflatable elements or channels 58 is disposed on the main graft 12 which may be inflated under pressure with an inflation material (not shown) through a main fill port 60 that has a lumen disposed therein in fluid communication with the network of inflatable channels 58. The inflation material may be retained within the network of inflatable channels 58 by a one way-valve (not shown), disposed within the lumen of the main fill port 60. The network of inflatable channels 58 may optionally be filled with a curable fluid in order to provide mechanical support to the main graft 12. The network of inflatable channels 58 may provide structural support to the main graft 12 when in an inflated state. The network of inflatable channels 58 may include a plurality of circumferential channels disposed about the main fluid flow lumen 18 or legs 20 and 26 of the main graft 12. The network of inflatable channels 58 may also include one or more inflatable cuffs 62 that are configured to seal to an inside surface of a patient's vessel. An inflatable element or cuff 62 is disposed on a proximal portion of the main graft 12 and has an outer surface that extends radially from a nominal outer surface of the main graft 12. The radial extension of the inflatable cuff 62 from the nominal outer surface of the main graft 12 may provide a seal against an inside surface of a blood vessel when the inflatable cuff 62 is in an inflated state. An interior cavity of the inflatable cuff 62 is in fluid communication with the interior cavity of the network of inflatable channels and may have a transverse dimension or inner diameter of about 0.040 inch to about 0.200 inch.

As shown in FIG. 1, two circumferential inflatable channels 64 are disposed on a distal portion of the ipsilateral graft extension 14 proximally of the ipsilateral connector ring 52. Although two circumferential inflatable channels 64 are shown, other embodiments may include one or more such inflatable channels 64 having a variety of configurations. The circumferential inflatable channels 64 can be inflated with an inflation material through an extension fill port 66. Some or all of the inflatable channels 58 and 64 (and similar channels of other components, such as, e.g., ipsilateral graft body section and contralateral graft body section described below) may be disposed circumferentially such as shown in the embodiment of FIG. 1. Alternatively, such channels 58 and 64 may be disposed in spiral, helical, or other configurations. Examples of channel configurations suitable for embodiments of the present invention are described further in commonly-owned pending U.S. patent application Ser. No. 10/384,103, filed Mar. 6, 2003 and entitled “Kink Resistant Endovascular Graft” to Kari et al., the entirety of which is incorporated herein by reference. All inflatable channel embodiments described herein as circumferential, may alternatively take on any of the aforementioned alternative configurations.

The inflatable cuff 62 and network of inflatable channels 58 and 64 may be filled during deployment of the graft with any suitable inflation material that provides outward pressure or a rigid structure from within the inflatable cuff 62 or network of inflatable channels 58 and 64. Biocompatible gases or liquids may be used, including curable polymeric materials or gels, such as the polymeric biomaterials described in issued U.S. Pat. No. 6,395,019 and pending U.S. patent application Ser. No. 09/496,231 filed Feb. 1, 2000, and entitled “Biomaterials Formed by Nucleophilic Addition Reaction to Conjugated Unsaturated Groups” to Hubbell et al. and pending U.S. patent application Ser. No. 09/586,937, filed Jun. 2, 2000, and entitled “Conjugate Addition Reactions for Controlled Delivery of Pharmaceutically Active Compounds” to Hubbell et al. and further discussed in commonly owned pending U.S. patent application Ser. No. 10/327,711, filed Dec. 20, 2002, and entitled “Advanced Endovascular Graft” to Chobotov, et al., each of which is incorporated by reference herein in its entirety.

The proximal anchor member 32 may be disposed on a proximal end of the main graft 12 and is secured to a proximal connector ring 68 which is at least partially disposed in a proximal portion of the main graft 12. The proximal connector ring 68 has connector elements 70 extending proximally from the proximal connector ring 68 beyond the proximal end of the main graft 12 in order to couple or be otherwise be secured to mating connector elements of the proximal anchor member 32. The proximal anchor member 32 may have a cylindrical or ring-like configuration with the element of the stent being preformed in a serpentine or sine wave pattern within the cylinder. The proximal anchor member may have a transverse dimension or diameter that allows for anchoring in a variety of aorta configurations. One embodiment of the proximal anchor member may have a transverse dimension or diameter of about 20.0 mm to about 40.0 mm. The elements of the proximal anchor member 32 may have a radial thickness of about 0.005 inch to about 0.040 inch. The width of the elements of the proximal anchor member 32 may be from about 0.01 inch to about 0.10 inch. Additional anchor members 72 may also be disposed at a proximal end of the proximal anchor member 32 having the same or similar features, dimensions or materials to those of the proximal anchor member 32. The terms “disposed in” and “disposed on” are used interchangeably throughout the specification. Such terms are meant to include a ring, stent, or other element being coupled to an interior surface of a layer, to an exterior surface of a layer, and between layers.

The anchor members 32, 36, 38 and 72 may have a variety of configurations that will be collapsible to a small transverse dimension or diameter for percutaneous or other types of delivery and be expandable to engage the inside surface of the patient's vasculature to provide anchoring to the vasculature and prevent or oppose axial movement of the anchor member or the graft section attached thereto. With specific regard to the ipsilateral and contralateral distal anchor members 36 and 38, the transverse dimension or diameter of these anchor members may be selected to reliably anchor in a wide range of iliac artery sizes. For example, embodiments of the ipsilateral and contralateral distal anchor members may have outer transverse dimensions or diameters of between about 15.0 mm to about 30.0 mm, more specifically, between about 20.0 mm and 25.0 mm. The anchor member embodiments 32, 36, 38 and 72 are configured as self-expanding anchor members having an undulating pattern and may be made from stainless steel, nickel titanium alloy or any other suitable material. The anchor members 32, 36, 38 and 72 may be configured to be balloon expandable or self-expanding and may also optionally include barbs 33 that are angled outwardly from the anchor members and are configured to engage tissue of the vessel wall and prevent axial movement of the anchor members once deployed. The proximal anchor member 32, additional anchor member 72 or other anchor members 36 and 38 may have the same or similar features, dimensions or materials to those of the stents described in commonly owned pending U.S. patent application Ser. No. 10/327,711, which was previously incorporated by reference. The proximal anchor member 32 and other anchor members 36, 38 and 72 may also be secured to a connector ring 52 and 68 in the same or similar fashion as described in the incorporated application above.

It may be useful for some embodiments of the main graft 12 to have a nominal axial length which is configured to allow the use of the main graft 12 in a wide variety of vascular morphologies with supplementation by one or more graft extensions 14. An endovascular graft 12 is normally chosen in order to have a proper fit to the patient's vasculature. For some endovascular graft indications, it is necessary to produce a large number of size variations of the graft system, or graft system 10 components, in order to accommodate the size and configuration variations of each patient's vasculature in order to achieve an acceptable fit of the graft system 10 within the patient's vasculature. This can be very costly and time consuming for the manufacturer of the endovascular graft system 10 and the hospitals which must maintain a comprehensive inventory of the devices. In addition, this may require an inconvenient amount of shelf space in the hospital operating room or catheter lab. In one embodiment, a main graft 12 has an axial length that is selected to allow anchoring of the proximal anchor member 32, ipsilateral distal anchor member 36 and optionally the contralateral distal anchor member 38 in a large cross section of patients having diverse physical size and vascular configurations. In this way, the need for customizing a graft system 10 for a particular patient or group of patients can be avoided.

In this embodiment, the axial length of the main graft 12, and particularly the axial distance or separation between the proximal anchor member 32 and ipsilateral distal anchor member 36, is selected to be just long enough to be properly anchored at both ends in the vasculature of a selected patient. The selected patient is the member of a group of patients who has the longest axial separation between the sealing point in the aorta just distal to the renal arteries and a proximal most viable anchor point in the iliac artery. In one embodiment for a particular patient group, the proximal end of the distal anchor member 36 is axially separated from the distal end of the proximal anchor member 32 by a length of at least about 11.0 cm, more specifically, at least about 15.0 cm, as indicated by the arrow 74 in FIG. 1.

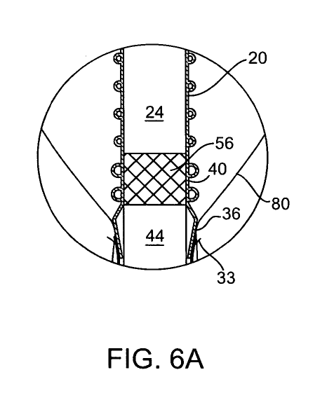

In an alternative method of sizing the main graft 12, the separation of the proximal anchor member 32 and ipsilateral distal anchor member 36 (and optionally the contralateral distal anchor member 38) is selected such that the separation, as indicated by arrow 74, is just long enough to span the separation between the renal arteries and the proximal most anchor point in the iliac artery or arteries of a patient, as indicated by arrow 75 in FIG. 6, below. This distance, indicated by arrow 75, is determined from the patient, in a selected group of patients, that has the longest such separation in the selected group of patients. In addition, for this embodiment, the separation indicated by arrow 74 must be shorter than the separation between the renal arteries and hypogastric artery or arteries 86 as indicated by arrow 77 in FIG. 6. The distance indicated by arrow 77 is determined from the patient, in the selected group of patients, that has the shortest such separation in the selected group of patients. In this way, it is possible to treat all members of a selected group of patients with a main graft 12 embodiment or embodiments which have a common separation between the proximal anchor member 32 and the ipsilateral distal anchor member 36 (and optionally the contralateral distal anchor member 38). Such an embodiment or embodiments can be anchored to the patient's aorta distal of the patient's renal arteries and anchored distally in the patient's iliac artery or arteries, without blocking either the renal arteries or hypogastric artery or arteries 86. Such an embodiment may have a separation, indicated by arrow 74, of about 11.0 cm to about 15.0 cm, specifically, about 12.0 cm to about 14.0 cm.

The careful sizing and configuring of the main graft 12 allows the use of a single main graft 12 embodiment or design to be adaptable to a wide range of patients when supplemented by one or more graft extensions 14. More specifically, a main graft 12 having a separation of about 12.0 cm to about 14.0 cm between the proximal anchor member 32 and the distal anchor member 36 can be properly anchored at both ends in a large percentage of potential patients. Once anchored, the fluid flow lumens 24 and 30 of the ipsilateral and contralateral legs 20 and 26 of the main graft 12 can then be sealed to the patient's iliac arteries with the deployment of graft extensions 14, if a seal is not created between the main graft and the patient's vasculature by initial deployment of the main graft 12. In addition, it is much easier to deploy graft extensions 14 into the ipsilateral and contralateral legs 20 and 26 once they have been anchored at their respective distal ends as there is no need to thread guidewires or other delivery devices into unanchored and shifting ports 22 and 28 of the ipsilateral and contralateral legs 20 and 26. Although the graft system 10 includes the option of using attachment elements 40, 42 and 56 to secure the graft extension 14 to the ipsilateral leg 20, this may not be necessary in most cases and an adequate seal and mechanical fixation of a graft extension 14 may be achieved with the use of a standard expandable member on the graft extension 14 instead of an attachment element 56.

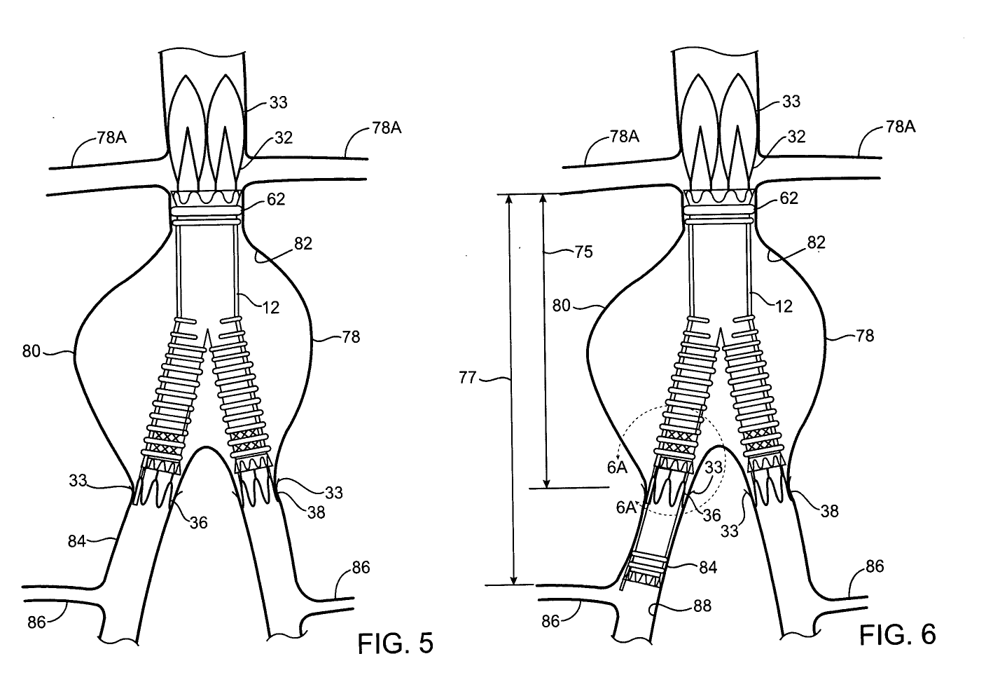

In use, a method of treating the vasculature of a patient includes providing the hybrid modular graft system 10 discussed above and illustrated in FIGS. 1-4. The main graft 12 is positioned within the patient's vasculature, specifically, the aorta 78, with the proximal anchor member 32 and proximal sealing cuff 62 positioned proximal of the aneurysm 80, as shown in FIG. 5. Other vessels of the patient's vasculature shown include the renal arteries 78A. The proximal anchor member 32 is then deployed and anchored to the patient's aorta 78. The proximal inflatable cuff 62 is filled with inflation material along with the network of inflatable channels 58 to seal to the inside surface 82 of the vessel. The ipsilateral distal anchor member 36 is positioned in an iliac artery 84 of the patient and deployed so as to anchor to the inside surface of the iliac artery 84 with the distal end of the graft extension disposed proximal of the hypogastric arteries 86. The graft extension 14 is positioned relative to the ipsilateral leg 20 of the main graft 12 such that the first attachment element 56 of the graft extension 14 is adjacent and longitudinally coextensive with the ipsilateral attachment element 40 of the ipsilateral leg 20 of the main graft 12. This position also provides for longitudinal overlap between the fluid flow lumen 44 of the graft extension 14 with the fluid flow lumen 24 of the ipsilateral leg 20, as shown in FIG. 6A. The ipsilateral attachment element 40 is then secured to the first attachment element 56 so as to extend the ipsilateral leg 20 of the main graft 12 with the inner lumen 24 of the ipsilateral leg 20 sealed to the inner lumen 44 of the graft extension 14. Thereafter, the distal anchor member 50 of the graft extension 14 may be deployed so as to anchor the distal anchor member 50 and distal end of the graft extension 14 to the patient's vasculature or iliac artery 84 as shown in FIG. 6. The deployment procedure carried out for the ipsilateral graft extension 14 may also be carried out with a contralateral graft extension (not shown) on the contralateral leg 26 of the main graft 12. In addition, the inflatable channels 58 and 64 of the main graft 12 and graft extension 14 may be inflated with an inflation material during the procedure. In one embodiment, the inflatable channels 58 and 64 are inflated after the proximal anchor member 32 has been deployed and anchored to the patient's aorta.

Deployment of the hybrid modular graft system 10 may be carried out by any suitable devices and methods, including techniques and accompanying apparatus as disclosed in commonly owned pending U.S. patent application Ser. No. 10/686,863, entitled “Delivery Systems and Methods for Bifurcated Endovascular Graft” to Chobotov et al., filed on Oct. 16, 2003, U.S. patent application Ser. No. 10/122,474, entitled “Delivery System and Method for Bifurcated Endovascular Graft” to Chobotov et al., filed on Apr. 11, 2002, U.S. patent application Ser. No. 10/419,312, entitled “Delivery System and Method for Expandable Intracorporeal Device” to Chobotov, filed Apr. 18, 2003, U.S. Pat. No. 6,733,521 to Chobotov et al., and U.S. Pat. No. 6,761,733 to Chobotov et al., the entirety of which are hereby incorporated herein by reference. In one specific deployment method embodiment, the main graft 12 is advanced in the patient's vessel 78, typically in a proximal direction from the ipsilateral iliac artery 84, to a desired site of deployment, such as the abdominal aorta, in a constrained state via a catheter or like device having a low profile for ease of delivery through the patient's vasculature 78. At the desired site of deployment, the proximal anchor member 32 of the main graft 12 is released from a constrained state and the proximal anchor member 32 is allowed to expand and secure a portion of the main graft 12 to the patient's vasculature 78. Thereafter, the network of inflatable channels 58 may be partially or fully inflated by injection of a suitable inflation material into the main fill port 60 to provide rigidity to the network of inflatable channels 58 and the main graft 12. In addition, a seal is produced between the inflatable cuff 62 and the inside surface of the abdominal aorta 82. Although it is desirable to partially or fully inflate the network of inflatable channels 58 of the main graft 12 at this stage of the deployment process, such inflation step optionally may be accomplished at a later stage if necessary. At this stage, the ipsilateral distal anchor member 36 (and optionally the contralateral distal anchor member 38) is released from a constrained state so as to deploy the anchor member 36 in the patient's iliac artery.

The graft extension 14 is then advanced into the patient's vasculature 78, again typically in a proximal direction from the ipsilateral iliac 84 in a constrained state via a catheter or like device until the first attachment element 56 is disposed within the ipsilateral attachment element 40 of the ipsilateral leg 20. The graft extension 14 is then released from the constrained state with the first attachment element 56 being pressed against and secured to the ipsilateral attachment element 40. The engagement of the ipsilateral attachment element 40 and first attachment element 56 is such that a seal is created between the elements 40 and 56. In addition, the engagement substantially prevents axial displacement or movement to separate the graft extension 14 from the ipsilateral leg 20. The inflatable channels 64 of the graft extension 14 may then be inflated to provide structural rigidity to the graft extension 14 and provide a seal between the circumferential inflatable channels 64 of the graft extension 14 and the inside surface 88 of the patient's iliac artery 84. Both the main fill port 60 and graft extension fill port 66 may include a valve (not shown), such as a one way valve, that allows the injection of inflation material but prevents the escape thereof. The same or similar procedure is carried out with respect to the deployment of the second or contralateral graft extension in the contralateral leg 26 of the main graft 12. The inflation channels 58 of main graft 12 and channels 64 of the graft extension 14 may be inflated in any sequence and in any number of partial steps until the desired level of inflation is achieved, to affect the desired clinical result. As such, the deployment and inflation sequence described above is but one of a large number of sequences and methods by which the embodiments of the present invention may be effectively deployed.

As discussed above, the main graft 12 embodiment of FIG. 1 need not be used with the graft extension 14 embodiment shown in FIG. 1. For example, main graft 12 could be used with a graft extension that has neither inflatable channels 64 nor an attachment element 56. Such a graft extension 104 embodiment is shown in FIG. 7, the use of which would obviate the need for the optional ipsilateral attachment element 40 and contralateral attachment element 42 on the ipsilateral leg 20 and contralateral leg 26 of the main graft 12, respectively. If a graft extension without attachment elements is used, it may be desirable to first deploy or release from a constrained state the distal end of the graft extension. In this way, the operator may use the patient's hypogastric artery or arteries to serve as a positioning reference point to ensure that the hypogastric arteries are not blocked by the deployment. Upon such a deployment, the proximal end of the graft extension may be deployed anywhere along the length of the ipsilateral leg 20. Also, although only one graft extension 14 is shown deployed on the ipsilateral side of the graft system 10, more graft extensions 14 may be deployed in graft extensions 14 already deployed in order to achieve a desired length extension of the ipsilateral leg 20 or contralateral leg 26. For example about 1 to about 5 graft extensions 14 may be deployed on either the ipsilateral or contralateral side of the graft system 10. Successive graft extensions 14 may be deployed within each other so as to longitudinally overlap fluid flow lumens 44 of successive graft extensions 14.

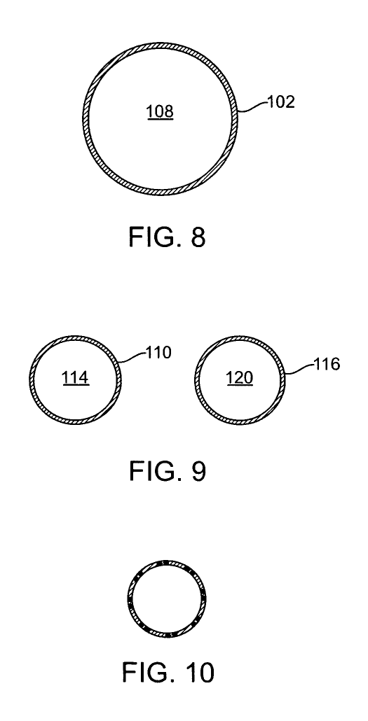

Referring to FIGS. 7-12, a non-inflatable hybrid modular graft system 100 is shown having a main graft 102 and an ipsilateral graft extension 104. The main graft 102 has a wall portion 106 that bounds a main fluid flow lumen 108 disposed therein. An ipsilateral leg 110 has a ipsilateral port 112 and an ipsilateral fluid flow lumen 114 that is in fluid communication with the main fluid flow lumen 108 and the ipsilateral port 112. A contralateral leg 116 has a contralateral port 118 and a contralateral fluid flow lumen 120 that is in fluid communication with the main fluid flow lumen 108 and the contralateral port 118. The main graft 102, ipsilateral leg 110 and contralateral leg 116 form a bifurcated “Y” shaped configuration with the main fluid flow lumen 108 of the main graft 102 typically having a larger transverse dimension and area than the fluid flow lumens 114 and 120 of either the ipsilateral leg 110 or contralateral leg 116. A proximal anchor member 122 is disposed at a proximal end of the main graft 102. An ipsilateral distal anchor member 124 is disposed on the distal end of the ipsilateral leg 110. A contralateral distal anchor member 126 is disposed on the distal end of the contralateral leg 116. The anchor members 122, 124 and 126 may optionally include barbs 33 which extend from the anchor members at angle configured to engage tissue of a vessel wall and prevent axial movement. In addition, the anchor members 122, 124 and 126 may also be self-expanding or balloon expandable.

The graft extension 104 has a fluid flow lumen 126 disposed therein which is sized and configured to be sealed in fluid communication with the fluid flow lumen 114 of the ipsilateral leg 110. Typically, an outside surface 128 of the graft extension 104 will be sealed to an inside surface 130 of the ipsilateral leg 110 of the main graft 102 when the graft extension 104 is deployed. A distal expansion member 132 is disposed on a distal end of the graft extension 104. The distal expansion member 132 may be in the form of the expandable member or stent. The distal expansion member 132 may be used to press the outside surface of the distal end of the graft extension 104 to the patient's vasculature. A proximal expansion member 134 is disposed on a proximal end of the graft extension 104. The proximal expansion member 134 may be in the form of the expandable member or stent. The proximal expansion member 134 may be used to press the outside surface of the proximal end of the graft extension 104 against an inside surface of the fluid flow lumen 114 of the ipsilateral leg 110.

The transverse dimension or diameter of the main fluid flow lumen 108 may be from about 15.0 mm to about 32.0 mm. The transverse dimension or diameter of the ipsilateral and contralateral fluid flow lumens 114 and 120 of the ipsilateral leg 110 and contralateral leg 116 may be from about 5.0 to about 20.0 mm. The main graft 102 and ipsilateral graft extension 104 may be made from polytetrafluoroethylene (PTFE) or expanded polytetrafluoroethylene (ePTFE). In particular, main graft 102 and graft extension 104 may comprise any number of layers of PTFE and/or ePTFE, including from about 2 to about 15 layers, having an uncompressed layered thickness of about 0.003 inch to about 0.015 inch. In general, the materials, features and dimensions of the main graft 102 and graft extension 104 may be the same as or similar to the materials, features and dimensions of the main graft 12 and graft extension 14 embodiments of FIG. 1. As with the embodiment discussed above, a second or contralateral graft extension (not shown) may have the same features as the ipsilateral graft extension 104 including a fluid flow lumen disposed therein and distal and proximal expansion members disposed at a distal end and proximal end of the second graft extension, respectively.

For some embodiments, the axial length of the main graft 102, and particularly the axial distance or separation between the proximal anchor member 122 and ipsilateral distal anchor member 124, may be selected by one or more of the criteria discussed above. In one embodiment for a particular patient group, the proximal end of the distal anchor member 124 is axially separated from the distal end of the proximal anchor member 122 by a length of about 11.0 cm to about 15.0 cm, more specifically, about 12.0 cm to about 14.0 cm, as indicated by the arrow 136 in FIG. 7. The length of the contralateral leg 116 is indicated by arrow 138 in FIG. 7. For one embodiment, the length of the legs 110 and 116 and can be from about 4.0 cm to about 10.0 cm. The careful sizing and configuring of the main graft 102 allows the use of a single main graft 102 embodiment or design to be adaptable to a wide range of patients when supplemented by one or more graft extensions 104. More specifically, a main graft 102 having separation of about 12.0 cm to about 14.0 cm between the proximal anchor member 122 and the distal anchor member 124 can be properly anchored at both ends in a large percentage of potential patients.

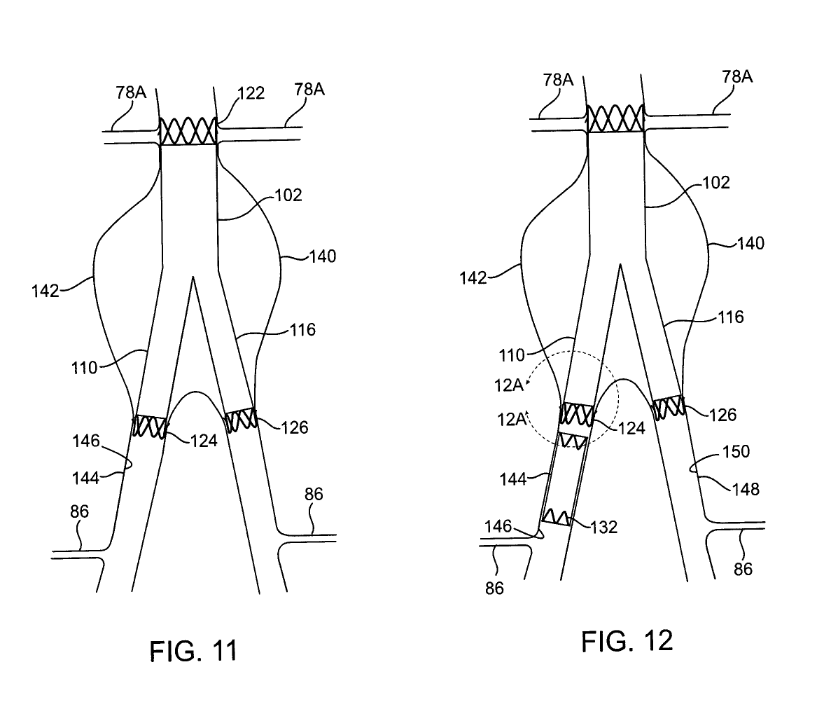

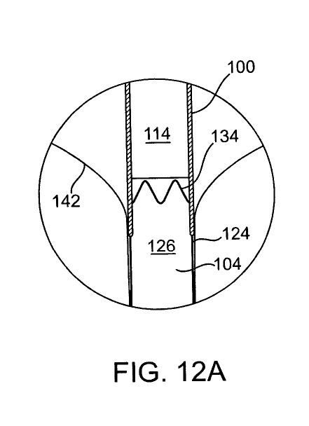

In use, a method of treating the vasculature of a patient includes providing the hybrid modular graft system 100 discussed above and illustrated in FIGS. 7-10. The main graft 102 is positioned within the patient's vasculature 140 with the proximal anchor member or stent 122 positioned proximal of the aneurysm 142, as shown in FIG. 11. The proximal anchor member 122 is then deployed and anchored to the patient's aorta. The ipsilateral distal anchor member 124 is positioned in an iliac artery 144 of the patient and deployed so as to anchor to the inside surface 146 of the iliac artery 144. The contralateral anchor member 126 is positioned in the contralateral iliac artery 148 of the patient and deployed so as to anchor the contralateral anchor member 126 to the inside surface 150 of the contralateral iliac artery 148. The graft extension 104 is then positioned relative to the ipsilateral leg 110 of the main graft 102 such that the proximal end of the graft extension 104 is disposed within the fluid flow lumen 114 of the ipsilateral leg 110. This position also provides for longitudinal overlap between the fluid flow lumen 126 of the graft extension 104 with the fluid flow lumen 114 of the ipsilateral leg 110, as shown in FIG. 12A. At this point, the proximal expansion member 134 of the graft extension 104 is released from a constrained state and allowed to expand and seal to an inside surface 130 of the fluid flow lumen 114 of the ipsilateral leg 110.

Thereafter, the distal expansion member 132 of the graft extension may be deployed or released from a constrained state so as to expand the distal end of the graft extension 104 against the inside surface 146 of the patient's vasculature 140 or iliac artery 144 as shown in FIG. 12. Alternatively, as discussed above, if a graft extension without attachment elements is used, it may be desirable to first deploy or release from a constrained state the distal end of the graft extension 104. In this way, the operator may use the patient's hypogastric artery or arteries to serve as a positioning reference point to ensure that the hypogastric arteries are not blocked by the deployment. Upon such a deployment, the proximal end of the graft extension 104 may be deployed anywhere along the length of the ipsilateral leg 20. The deployment procedure carried out for the ipsilateral graft extension 104 may also be carried out with a contralateral graft extension (not shown) on the contralateral leg of the main graft. Also, although only one graft extension 104 is shown deployed on the ipsilateral side of the graft system 100, more graft extensions 104 may be sequentially deployed in graft extensions 104 already deployed in order to achieve a desired length extension of the ipsilateral leg 110 or contralateral leg 116. For example about 1 to about 5 graft extensions 104 may be deployed on either or both the ipsilateral or contralateral side of the graft system 100. Successive graft extensions 104 may be deployed within each other so as to longitudinally overlap fluid flow lumens 126 of successive graft extensions 104. Moreover, while graft extension 104 embodiment of FIG. 7 is shown in conjunction with main graft 102 of FIG. 7, one or more graft extension 104 embodiments may also be used in conjunction with main graft 12 embodiment shown in FIG. 1, as discussed above.

While particular forms of embodiments of the invention have been illustrated and described, it will become apparent that various modifications may be made without departing from the spirit and scope of the invention. Accordingly, it is not intended that the invention be limited by the foregoing exemplary embodiments.

What is claimed is:

1. A hybrid modular endovascular graft system, comprising:
a main graft having a main fluid flow lumen therein, a distal leg of the main graft having a fluid flow lumen therein, a proximal anchor member disposed at a proximal end of the main graft and a distal anchor member disposed on a distal portion of the distal leg, the distal anchor member being axially separated from the proximal anchor member by a length of about 12.0 cm to about 14.0 cm; and
a graft extension having a fluid flow lumen disposed therein with the fluid flow lumen of the graft extension sealed to and in fluid communication with the fluid flow lumen of the distal leg.

2. The graft system of claim 1 wherein the fluid flow lumen of the graft extension is overlapped with the fluid flow lumen of the distal leg of the main graft.

3. The endovascular graft system of claim 1 wherein the proximal anchor member and distal anchor member comprise expandable stents.

4. The endovascular graft system of claim 1 wherein the graft extension further comprises a distal anchor member disposed at a distal end of the graft extension.

5. The endovascular graft system of claim 1 wherein the main graft further comprises a network of inflatable channels distributed over a main graft body section to provide structural rigidity and support to the main graft when the network of inflatable channels are in an inflated state.

6. The endovascular graft system of claim 5 wherein wall portions of the main graft and graft extension comprise layered ePTFE.

7. The graft system of claim 1 wherein the main graft comprises a bifurcated graft and further comprises a second distal leg having a fluid flow lumen in fluid communication with the main fluid flow lumen and a second distal anchoring member disposed on a distal portion of the second distal leg.

8. The graft system of claim 7 further comprising a second graft extension having a fluid flow lumen disposed therein which is in fluid communication with the fluid flow lumen of the second distal leg.

9. A method of treating the vasculature of a patient, comprising:
providing a graft system, comprising:
a main graft having a main fluid flow lumen therein, a distal leg having a fluid flow lumen therein, a proximal anchor member disposed at a proximal end of the main graft and a distal anchor member disposed on a distal portion of the distal leg, the distal anchor member being axially separated from the proximal anchor member by a distance of about 12.0 cm to about 14.0 cm, and
a graft extension having a fluid flow lumen disposed therein with the fluid flow lumen of the graft extension sealable to the fluid flow lumen of the distal leg;
positioning the main graft within the patient's vasculature and anchoring the proximal anchor member in the patient's aorta and anchoring the distal anchor member in an iliac artery of the patient;
positioning the graft extension relative to the main graft such that the fluid flow lumen of the graft extension is sealed with the fluid flow lumen of the distal leg.

10. The method of claim 9 wherein the graft extension further comprises a distal anchor member disposed at a distal end of the graft extension and further comprising anchoring the distal anchor member of the graft extension to the patient's vasculature.

11. The method of claim 9 wherein the main graft further comprises a network of inflatable channels distributed over a main graft body section and further comprising inflating the network of inflatable channels.

12. The method of claim 9 wherein the main graft comprises a bifurcated graft and further comprises a second distal leg having a fluid flow lumen in fluid communication with the main fluid flow lumen and the graft system further comprises a second graft extension having a fluid flow lumen disposed therein and further comprising anchoring the second distal anchor member and sealing the fluid flow lumen of the second graft extension to the fluid flow lumen of the second distal leg.

13. A hybrid endovascular graft, comprising:
a main graft having a main fluid flow lumen therein, a distal leg of the main graft having a fluid flow lumen therein, a proximal anchor member disposed at a proximal end of the main graft and a distal anchor member disposed on a distal portion of the distal leg, the distal anchor member being axially separated from the proximal anchor member by a length of about 11.0 cm to about 15.0 cm.

14. The hybrid modular endovascular graft of claim 13 wherein the distal anchor member is axially separated from the proximal anchor member by a length of about 12.0 cm to about 14.0 cm.

15. A method of sizing a main graft of a hybrid modular endovascular graft system, comprising:
selecting a group of patients to be treated;
sizing the axial separation of the proximal anchor member and ipsilateral distal anchor member of a main graft such that the axial separation is no shorter than the separation between the renal arteries and the proximal most anchor point in the iliac artery or arteries of a patient who has the longest such separation, and no longer than the separation between the renal arteries and hypogastric artery or arteries of a patient who has the shortest such separation.

16. The method of claim 15 wherein the axial separation of the proximal anchor member and ipsilateral distal anchor member of a main graft is selected to be about 11.0 cm to about 15.0 cm.

17. The method of claim 16 wherein the axial separation of the proximal anchor member and ipsilateral distal anchor member of a main graft is selected to be about 12.0 cm to about 14.0 cm.
